(19) United States
(12) Patent Application Publication
(10) Pub. No.: US 2007/0166252 A1
(43) Pub. Date: Jul. 19, 2007

(54) Method of treating skin requiring tattoo removal

(76) Inventors: Judy Hattendorf, Marina Del Ray, CA (US); Steve Carlson, San Mateo, CA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/648,290

(22) Filed: Dec. 29, 2006

Related U.S. Application Data
(60) Provisional application No. 60/755,357, filed on Dec. 30, 2005.

(51) Int. Cl. A61K 8/00; A61K 8/02; A61K 8/18; A61Q 5/08

(52) U.S. Cl. 424/62; 424/401

(57) ABSTRACT

A treatment regimen for treating skin subject to tattoo removal procedures involves the application of supplemental composition(s) such as preparatory composition(s), protective composition(s), and combinations thereof, and a corrective composition.

### CROSS REFERENCE TO RELATED APPLICATION

This Application claims priority benefit of U.S. Provisional Application No. 60/755,357 filed Dec. 30, 2005 the entire disclosure of which is incorporated herein by this reference.

### BACKGROUND

1. Technical Field

This disclosure relates to the sequential topical application of compositions in a prescribed fashion to minimize the adverse effects of tattoo removal. The disclosure further relates to the pre and post application of corrective compositions and one or more supplementary compositions to skin in association with tattoo removal. Kits containing corrective compositions and supplementary compositions for use in connection with tattoo removal are also described.

2. Background of Related Art

Recently, tattoos have become increasingly popular. For instance, the tattoo industry ranked sixth in a 1996 study that estimated the growth rates of various industries. The practice of permanent tattooing has become so widespread that it is now estimated that as many as 30 million people in the Western world have at least one tattoo.

Some who choose to be tattooed may at some later date regret the decision. The spirit, motivation, and/or circumstances that compelled one to obtain a tattoo can fade. In some cases, a tattoo that was appropriate or desirable at one station of life may be less appropriate or less desirable at a later date. Depending upon the nature and extent of the tattoo image, an unwanted tattoo may be a nuisance, a source of embarrassment, or even a source of social stigmatization. Perhaps as a direct result of the increased popularity of tattoos, interest in tattoo removal also is increasing. Market estimates suggest that in the year 2000 as many as 410,000 people underwent a tattoo removal procedure. As the recently tattooed population ages, it is expected that the number of those seeking removal of a tattoo will increase.

Current treatment options for tattoo removal include a variety of lasers, dermabrasion, salabrasion, surgical excision, and cryotherapy. Although some treatments may be effective, they may be expensive, time consuming, and painful. In some cases, such treatments also may result in cosmetically undesirable scarring.

Thus, there remains room for improvement in tattoo removal techniques, and especially to minimize, reduce, or eliminate the undesirable side effects and/or the need for multiple treatments.

### SUMMARY

Skin requiring tattoo removal is pre-treated in accordance with the present disclosure by preconditioning skin by the sequential topical application of one or more corrective compositions, and one or more supplementary compositions in a morning regimen; followed by the sequential topical application of one or more corrective compositions, including tretinoin, and one or more supplementary compositions in an evening regimen. Such preconditioning by the sequential application of such compositions may minimize, reduce, or eliminate the undesirable side effects and/or the need for multiple tattoo removal treatments.

Optionally, after a tattoo removal process is performed on the preconditioned skin, the skin may be post-treated by another sequential topical application of one or more corrective compositions, and one or more supplementary compositions in a morning regimen; followed by the sequential topical application of one or more corrective compositions, including tretinoin, and one or more supplementary compositions in an evening regimen. Post-treatment of preconditioned skin may minimize, reduce, or eliminate the undesirable side effects of tattoo removal such as reactions or complications like post-inflammatory hyperpigmentation, erythema (redness), acne and scarring. Post-treatment may also reduce the need for multiple tattoo removal treatments.

In addition, dermatological treatment regimens in accordance with the present disclosure may improve characteristics of a user's skin. The regimens include the application of one or more corrective compositions and the application of one or more supplementary compositions. Suitable corrective compositions include, for example, compositions which help to repair damage to the deeper layers of skin, or stable corrective compositions which contain one or more active ingredients sensitive to oxidation that remain stable for three years at room temperature. Suitable supplementary compositions include, for example: preparatory compositions which make skin more receptive to the corrective compositions; or protective compositions which further protect skin against damage from harmful UVA and UVB rays. Depending on the nature of the one or more supplementary compositions, they may be applied before, after, or both before and after application of the corrective composition.

In embodiments, the present disclosure is directed towards kits for pre-treating and post-treating skin subject to a tattoo removal procedure containing both one or more stable corrective compositions and one or more supplementary compositions.

These and other aspects of this disclosure will be evident upon reference to the following detailed description.

### DETAILED DESCRIPTION OF PREFERRED EMBODIMENTS

Treatment regimens in accordance with this disclosure include the sequential steps of: pre-treating the surface of skin in need of a tattoo removal procedure; performing a tattoo removal procedure; and optionally post-treating the skin. The pre-treatment and post-treatment may include the sequential topical application of treatment compositions in a prescribed fashion.

The first step of the present method is pre-treatment. The pre-treatment step of the treatment regimen of the present disclosure is designed for pre-conditioning the skin to rendering the area of skin to be treated more receptive and responsive to a tattoo removal procedure. For example, skin that is oily or dirty can be preconditioned to a healthier more hygienic state. It has been found that preconditioning skin by cleaning, toning, exfoliating, and/or using corrective compositions in accordance with the present disclosure may reduce adverse effects of a tattoo removal procedure. Moreover, the sequential topical application of treatment compositions in accordance with the present disclosure may contribute to tattoo removal procedures by providing excellent aesthetic results with reduced recovery periods, while markedly improving rejuvenation goals and aesthetic expectations.

Thus, skin in need of tattoo removal treatment is pre-treated by preconditioning skin by the sequential topical application of one or more corrective compositions, and one or more supplementary compositions in a morning regimen; followed by the sequential topical application of one or more corrective compositions, including tretinoin, and one or more supplementary compositions in an evening regimen.

As used herein the word “treat,” “treating” or “treatment” refers to using the compositions of the present disclosure prophylactically to prevent outbreaks of undesirable dermatological symptoms, or therapeutically to ameliorate an existing undesirable dermatological condition, and/or extend the duration of the aesthetic benefit of a tattoo removal procedure, or reduce the frequency of repeated tattoo removal procedures.

Pre-treatment regimens in accordance with the present disclosure improve skin characteristics through sequential application of pre-selected skin care compositions to the skin of a user prior to the tattoo removal procedure. As used herein the word “corrective composition” refers to using the compositions of the present disclosure which have an active ingredient for treating any undesirable dermatological condition. Application of the corrective composition in combination with the one or more supplementary compositions provides improved effectiveness of the corrective composition compared to application of the corrective composition alone. As used herein the word “supplementary composition” refers to using compositions of the present disclosure which do not have active ingredient for treating undesirable dermatological conditions, however when used in conjunction with a corrective composition produce a beneficial effect.

The supplementary composition can be, for example, a preparatory composition which makes the skin of the user more receptive to the corrective composition. Alternatively, the supplementary composition may be a protective composition which protects skin against damage from harmful UVA and UVB rays.

Depending on the exact nature of the supplementary compositions employed, the supplementary composition may be applied before or after application of the corrective composition. For example, where the supplementary composition is a preparatory composition, the supplementary composition is applied before the corrective composition. Where the supplementary composition is a protective composition, the supplementary composition is applied after the corrective composition. In particularly useful embodiments, one or more supplementary compositions can advantageously be applied both before and after application of the corrective composition.

In embodiments, pre-treatment regimens in accordance with the present disclosure include a first treatment, such as in the morning hours, and a second treatment, such as in the evening hours. Both the first and second treatments include the topical application of one or more corrective compositions, along with one or more supplemental compositions. It should, of course be understood that the supplementary compositions used in the first treatment can be different from the supplementary compositions applied in the second treatment. Likewise, it should be understood that the corrective compositions used in the first treatment can be different from the corrective compositions applied in the second treatment.

In embodiments, the first treatment occurs in the morning hours and includes the application of an effective amount of one or more preparatory compositions (e.g., foaming gel, and toner) followed by application of an effective amount of one or more corrective compositions (e.g., exfoliator, and/or one or more hydroquinone compositions) followed by the application of an effective amount of one or more protective compositions (e.g., sun protector). In this embodiment, the second treatment occurs in the evening hours and includes the topical application of an effective amount of one or more preparatory compositions (e.g., foaming gel, and toner) followed by application of an effective amount of one or more corrective compositions (e.g., hydroquinone and tretinoin compositions). In embodiments, tretinoin is used either alone or in combination with other corrective compositions during the evening treatment.

Materials suitable for use as pre-treatment composition include corrective compositions and supplementary compositions pre-selected to clean, tone, exfoliate, treat or precondition skin in need of a tattoo removal procedure. Non-limiting examples of pre-treatment compositions are listed below and include supplementary compositions such as cleanser compositions, toner compositions, and exfoliant compositions. Other suitable pre-treatment compositions include corrective compositions such as stable corrective compositions and stimulating corrective compositions. The pre-treatment compositions are categorized in various classes however this classification is not intended to limit the pre-treatment compositions in any way to only to those pre-treatment compositions belonging to the categories herein mentioned. Moreover, as described below, the same or different pre-treatment compositions can be used as post-treatment compositions in accordance with the present disclosure.

Supplementary Compositions

Non-limiting examples of supplementary compositions which may be combined with the corrective compositions of this disclosure are listed below.

Suitable supplementary compositions are categorized in various classes (e.g. preparatory compositions and protective compositions) however this classification is not intended to limit the supplemental compositions in any way to only those compositions belonging to the categories herein mentioned.

Preparatory Compositions

Throughout the pre-treatment regimen of the present disclosure, skin improvement may be slowed or worsened by skin becoming dirty or oily throughout the day and night. Dirt and oil clog pores and slow the corrective compositions from contacting the inner layers of skin. One class of supplementary compositions that may be combined with the corrective compositions of the present disclosure is preparatory compositions which make skin more receptive to the corrective step.

Thus, the pre-treatment regiment of the present disclosure includes the step of preparing skin to make it more receptive to the corrective step by applying preparatory compositions. Suitable preparatory compositions include cleansers, foaming gels, toners, and combinations thereof, which may be applied to the skin in the morning or evening portion of the treatment regimen.

The cleanser is applied to skin in amounts that provide the benefit to the skin of the user, such as in an amount sufficient to remove dirt and oil from the skin. Generally, the cleansers are soap-free and include water, detergent, surfactant, humectants, skin conditioning agent, PH adjustor, extracts, preservatives, fragrance and colorant, however, any cleaner suitable for removing dirt and oil from skin may be used. One commercially available cleanser is Obagi Nu-Derm® gentle cleanser available from OMP, Inc. of Long Beach, Calif. The Obagi Nu-Derm® cleanser contains a combination of water, cocamidopropyl betaine, sodium lauroyl oat amino acids, sodium laureth sulfate, glycerin, aloe barbadensis gel, glycerth-7, apricot triethanolamine, sage extract, borage extract, phenoxythanol, methylparaben, propylparaben, ethylparaben, butylparaben, saponins, fragrance, and colorant.

Optionally, a foaming gel may be applied as one of the preparatory compositions in amounts that provide the benefit to the skin of the user, such as in an amount sufficient to remove dirt, oil and/or impurities to clean skin and leave it more receptive to treatment. Generally, foaming gels include water, detergent, surfactant, humectants, skin conditioning agent, PH adjustor, extracts, preservatives, fragrance and colorant, however any foaming gel may be applied that cleans the skin by removing dirt and/or oil. One commercially available foaming gel is Obagi Nu-Derm® foaming gel available from OMP, Inc. of Long Beach, Calif. The Obagi Nu-Derm® foaming gel contains a combination of water, sodium lauryl oat amino acids, cocamidopropyl betaine, sodium laureth sulfate, aloe barbadensis gel, alfalfa extract, borage extract, sodium chloride, xantham gum, saponins, phenoxythanol, methylparaben, propylparaben, ethylparaben, butylparaben, fragrance and colorant.

Optionally, toner may be applied as a preparatory composition in amounts that provide the benefit to the skin of the user, such as in an amount sufficient to hydrate and tone skin while reducing the pH. Toner also may help remove dirt, oils, and grime without overly drying out sensitive skin. Generally, toners include water, skin conditioner, astringent, minerals, moistening agent, vitamins and complexes thereof, anti-microbial, cleanser, extract, surfactant, anti-irritant, fragrance and colorant; however any commercially available skin toner may be used. One commercially available toner is Obagi Nu-Derm® toner available from OMP, Inc. of Long Beach, Calif. The Obagi Nu-Derm® toner contains a combination of water, aloe barbadensis gel, witch hazel distillate, potassium alum, sodium PCA, panthenol, DMDM hydantion, polysorbate 80, allantoin, sage extract, calendula officinalis extract, saponins, fragrance, and colorant.

During the treatment regimen, the preparatory composition(s) can advantageously be applied to damp skin of the face and neck with moistened fingertips. The face and neck of the user can be rinsed with warm water after application of the preparatory composition(s).

Protective Compositions

Skin improvement may be slowed or worsened by sunrays which may cause pigmentation and dryness. Accordingly, protective compositions are one class of supplementary compositions that optionally may be combined with the corrective compositions in the treatment regimens of the present disclosure to alleviate sun damage or dryness.

Suitable protective compositions include any composition capable of reducing skin damage, darkening, or dryness. In embodiments, protective compositions include sun block to screen out ultraviolet light rays. In embodiments, suitable protective compositions include creams are moisturizers formulated to help control dryness.

One suitable commercially available protective composition is Obagi Nu-Derm® Sunblock, from OMP, Inc. of Long Beach, Calif. This protective composition provides broad-spectrum sun protection and can advantageously be applied every morning as part of a treatment regimen in accordance with this disclosure. The formulation is made of octinoxate, zinc oxide, butylparaben, cetearyl alcohol, citric acid, C13-14 isoparaffin, diethanolamine cetyl phosphate, disodium edetate, ethylparaben, isobutylparaben, isopropyl palmitate, laureth-7, methylparaben, octyl stearate, phenoxyethanol, polyacrylamide, polyether-1, polysorbate 60, propylparaben, purified water, sodium hydroxide, and triethoxycaprylylsilane.

Another suitable commercially available protective composition is Obagi Nu-Derm® Physical UV Block SPF 32. This composition contains zinc oxide USP, beeswax, butylene glycol, cetyl dimethicone, cetyl PEG/PPG-10/1 dimethicone, dimethicone, disodium EDTA, glycereth-26, hydrogenated castor oil, isopropyl palmitate, methylparaben, octyl stearate, propylparaben, purified water, sodium propylparaben, purified water, sodium chloride, triethoxycaprlylsilane, tocopherol acetate, and willowherb extract.

Other suitable commercially available protective compositions include Obagi Nu-Derm® Healthy Skin Protection (SPF 35) and Obagi Nu-Derm® Eye Cream.

Corrective Compositions

Non-limiting examples of corrective compositions which may be combined with the supplementary compositions of this disclosure are listed below.

Suitable corrective compositions are categorized in various classes (e.g. stimulating corrective compositions and stable protective compositions) however this classification is not intended to limit the corrective compositions in any way to only those compositions belonging to the categories herein mentioned. In fact, where necessary all ingredients used in the stable corrective compositions may be utilized to make the stimulating corrective compositions regardless of stability achieved.

Stimulating Corrective Compositions

Throughout the treatment regimen of the present disclosure, skin may be, among other things, regenerated by contacting the skin with one or more stimulating corrective compositions. Such compounds include those capable of the gradual bleaching of hyper-pigmented skin conditions such as chloasma melasma, freckles, sensile lentigines, and other unwanted areas of melanin hyper-pigmentation. Thus, suitable stimulating corrective compositions include, but are not limited to compositions having one or more active ingredients which help to repair damage to the deeper layers of skin, such as blenders, tretinoin compositions, retin-A compositions, and combinations thereof.

The corrective compositions can be applied to the skin in amounts that provide the benefit to the skin of the user, such as in an amount sufficient to repair damage to the deeper layers of skin. Typically corrective compositions are applied to the skin in the two treatments per day, such as a morning (a.m.) and evening (p.m.) treatment. It should of course be understood that applying corrective compositions in one treatment is also possible, especially where the active ingredient is potent, such as tretinoin.

One corrective composition is a blender which promotes pigmentation correction at the cellular level promoting even skin color. Generally blenders include skin lightening agent such as hydroquinone, preservative, chelating agent, emulsifier, humectant, pH adjuster, antioxidant, emollient, reducing agent and water. Moreover, blenders with improved stability as those described below may also be used for correcting the skin in accordance with this disclosure.

One commercially available blender is Obagi Nu-Derm® Blender available from OMP, Inc. of Long Beach, Calif. The Obagi Nu-Derm® Blender contains a combination of hydroquinone USP 40 mg/gm in a base of purified water, glycerin, cetyl alcohol, PPG-2 myristyl ether propionate, sodium lauryl sulfate, TEA-salicylate, lactic acid, phenyl trimethicone, tocopheryl acetate, sodiummetabisulfite, ascorbic acid, methylparaben, saponins, disodium EDTA, BHT and propylparaben.

Other suitable corrective compositions include retinoid containing compositions applied in amounts sufficient to provide benefit to the skin, such as medically prescribed tretinoin. Tretinoin skin preparations are a family of drugs all similar to Vitamin A available in gel or cream form. Tretinoin can advantageously be used in combination with alpha hydroxyacid preparations. The inclusion of a tretinoin corrective composition in the present treatment regimen may aid in keratinocyte activity regulation, mitosis, repairing damaged DNA, blood vessel formation such as angiogenesis, and the creation of a soft epidermis.

The incorporation of retinoid containing corrective composition into the sequential treatment regimen of the present disclosure can promote a smoother less wrinkled skin and can be effective in treating sun damage, wrinkling, hyperpigmentation and facial roughness. Although not wishing to be bound by this disclosure, it is believed that tretinoin passes through the skin cell membranes to the nucleus wherein it binds to nuclear receptors and regulates transcription of genes that mediate the rate of cell division and turnover, cell differentiation and formulation of new healthy collagen and the repair of elastin. As a result skin can be firmer from the collagen formation as well as more flexible from the repair of elastin.

Tretinoin also increases the formation of normal keratinocytes (cells making up about 90% of the epidermis) and fibroblasts (connective tissue cells which secrete an extracellular matrix rich in collagen and other macromolecules), decreases melanocyte activity (which offers better resistance to external injury and inflammation) and is found to improve angiogenesis (the formation of new blood vessels that increase skin circulation).

Suitable tretinoin compositions for use with the treatment regimen of the present disclosure utilize a medically prescribed tretinoin medication such as, 0.05 and 0.1 Tretinoin (generic). Suitable tretinoin compositions are commercially available under a variety of trade names. In embodiments, the retinoid containing stimulating composition used in the treatment regimen of the present disclosure is an oil-in-water emulsion, such as commercially available tretinoin creams containing 0.05% or 0.1% actives.

Another suitable corrective composition is Obagi Nu-Derm® Sunfader, from OMP, Inc. of Long Beach, Calif. Each gram of Obagi Nu-Derm® Sunfader contains hydroquinone, octinoxate, and oxybenzone 5.5% in a base of purified water, cetyl alcohol, glycerin, sodium lauryl sulfate, stearyl alcohol, tocopheryl acetate, ascorbic acid, sodium metabisfulfite, disodium EDTA, methylparaben, saponins, propylparben, BHT and butylparaben.

Another suitable commercially available corrective composition is Obagi Nu-Derm® Clear from OMP, Inc. of Long beach, Calif. One gram of Obagi Nu-Derm® Clear contains hydroquinone in a base of purified water, cetyl alcohol, glycerin, sodium lauryl sulfate, stearyl alcohol, tocopheryl acetate, ascorbic acid, sodium metabisulfite, lactic acid, saponins, disodium EDTA, methylparaben, BHT, propylparaben and butylparaben.

Other corrective compositions include exfoliating lotion to smooth and tone rough or damaged skin. Suitable exfoliators include Obagi Nu-Derm® Exfoderm and Obagi Nu-Derm® Exfoderm Forte. Obagi Nu-Derm® Exfoderm typically contains water, ethoxydiglycol, phytic acid, glycerin, cetearyl alcohol, glyceryl stearate, PEG-100 stearate, canola oil, isohexadecane, magnesium aluminum silicate, potassium cetyl phosphate, cetyl alcohol, bis-diglyceryl polyacyladipate-2, dimethicone, polysorbate 0, PEG-150 stearate, steareth-20, xanthan gum, glycereth-7, tocopheryl acetate, saponins, phenoxyethanol, methylparaben, propylparaben, butyparaben, ethylparaben, isobutylparaben. Obagi Nu-Derm® Exfoderm Forte typically contains purified water, glycolic acid, emulsifying wax, triethanolamine, glycerin, lactic acid, caprylic/capric triglyceride, kalaya oil, stearic acid, cetyl alcohol, dimethicone, methylparaben, propylparaben, saponins.

Stable Corrective Compositions

In any topical treatment regimen, instability of the topical composition containing the active may cause degradation of the active requiring application of unnecessarily large amounts of the active ingredient and of potentially irritating degradation by-products to the skin. It has been found that these problems can be eliminated or reduced by the use of corrective composition having three years of stability at room temperature.

Accordingly, in embodiments the present disclosure relates to stable active containing corrective compositions. These stable corrective compositions can be made, for example, by the methodology described in this disclosure copending application Ser. No.11/291,400, the entire disclosure of which is incorporated herein by this reference; however, any method of making the corrective compositions may be employed so long as they achieve the desired stability. Thus, the stable corrective compositions are formulated, manufactured and packaged in accordance with this disclosure in a manner which enables the composition to remain in the package without discoloring. As used herein the term “stable” means that the composition when in a closed container remains within the tolerances and limits set forth in US Pharmacopeia and/or the US FDA guidelines or monographs for compositions containing any particular active ingredient or combination of active ingredients. The entire US Pharmacopeia and collection of US FDA guidelines or monographs for compositions containing any particular active ingredient or combination of active ingredients are too voluminous to present in their entirety herein and thus are instead incorporated in their entirety by this reference. With respect to topical compositions, the tolerances and limits are frequently presented relative to the labeled amount. As one illustrative example, for hydroquinone cream, the acceptable tolerance is not less than 94.0 percent and not more than 106.0 percent of the labeled amount of C₆H₆O₂. As another illustrative example, for tretinoin cream, the acceptable tolerance is not less than 90.0 percent and not more than 130.0 percent of the labeled amount of C₂₀H₂₈O₂. Those skilled in the art will readily be able to identify the tolerances and limits for other compositions containing other active ingredients.

As those skilled in the art will appreciate, the container-liner-closure system used to store the composition will affect the stability of the active ingredient. It should be understood that a composition need not be stable in all containers to be stable in accordance with this disclosure. Stability in at least one type of container is sufficient for a composition to be stable as that term is used herein.

In embodiments, stable corrective compositions in accordance with the present disclosure can be stable for at least three years at room temperature. Stability of the present compositions can be evaluated through accelerated stability studies. In these studies, the packaged composition is maintained at an elevated temperature for a period of time after which it is examined. The exposure to elevated temperatures for a given period correlates to a correspondingly longer period of time at room temperature. Thus, for example, if a product remains within the required tolerances and limits when maintained for a period of 12 weeks at a temperature of 40° C. and 12 months further at room temperature, one can conclude that the product has a shelf life of greater than two and up to three years at room temperature. Those skilled in the art will envision other testing to confirm the stability of the products described herein.

Tattoo Removal Techniques

The second step of the present methods is performing a tattoo removal procedure on preconditioned skin. Any tattoo removal procedure may be employed. Suitable tattoo removal procedures are within the purview of those skilled in the art. Tattoo removal may be achieved, for example, through dermabrasion (where skin is “sanded” to remove the surface and middle layers), cryosurgery (where the area is frozen prior to its removal) excision (where a surgeon removes the tattoo with a scalpel and closes the wound with sutures or, in some cases involving large tattoos, employing a skin graft from another part of the body); and laser treatment (where the type of laser used to remove a tattoo depends on the tattoo's pigment colors with yellow and green being the hardest colors to remove; blue and black being the easiest). Three lasers developed specifically for use in tattoo removal using a technique known as Q-switching, (which refers to the laser's short, high-energy pulses) are the Q-switched Ruby, the Q-switched Alexandrite, and the Q-switched Nd: YAG. Illustrative examples of tattoo removal procedures include, but are not limited to, the methods disclosed in: Published U.S. patent application Ser. No. 20040181211A1 entitled Method Of Tattoo Removal; U.S. Pat. No. 6,773,698 entitled Tattoo Removal; Published European Patent Application No. EP1514574A2 entitled Scar-Free Tattoo Removal; and Published European Patent Application No. EP0616795A1 entitled Apparatus For Tattoo Removal. The entire disclosure of each of the aforementioned patents and published applications is incorporated herein by this reference.

Tattoo removal may also be accomplished through chemical means. Suitable chemical tattoo removal methods are within the purview of those skilled in the art. In embodiments, the chemical tattoo removal procedure is achieved via a chemical peel. Suitable chemical peel products are commercially available. One such product is the Obagi Blue Peel System from OMP, Inc. of Long beach, Calif which includes a non-ionic blue base with glycerin for use in performing a tricloracetic (TCA) peel.

The tattoo removal step will typically be performed in accordance with techniques known in the art by a physician.

POST-TREATMENT

Optionally, the treatment regimen can include post-treating the pre-conditioned/treated skin. Typically, the type of tattoo removal procedure performed on the skin treatment area will dictate the type of post-treatment compositions to be applied. For example, the procedure can be varied depending on the apparatus used by the dermatologist in performing the tattoo removal procedure. Furthermore, the post-treatment can include repeating the pre-treatment steps described above with the same or different pre-treatment compositions including any supplementary compositions and corrective compositions described above.

It should be noted that post-treatment can comprise not just a single application of a single corrective or supplementary composition but can be a sequentially applied treatment. For example, multiple supplementary compositions can be used as well as multiple corrective compositions. Thus the classification as a post-treatment composition is not intended to limit the post-treatment compositions in any way to only those post treatment compositions mentioned herein.

In embodiments, after treating the preconditioned skin with a tattoo removal procedure, skin is post-treated by another sequential topical application of one or more corrective compositions, and one or more supplementary compositions in a morning regimen; followed by the sequential topical application of one or more corrective compositions, including tretinoin, and one or more supplementary compositions in an evening regimen. Post-treatment of preconditioned skin enhances the benefits of the tattoo removal procedure by limiting adverse events associated with tattoo removal procedures.

In embodiments, the first post-treatment occurs in the morning hours and includes the application of an effective amount of one or more preparatory compositions (e.g., gentle cleanser, and toner) followed by application of an effective amount of one or more corrective compositions (e.g., Obagi Nu-Derm® Exfoderm, and/or one or more hydroquinone compositions such as Obagi Nu-Derm® Clear) followed by the application of an effective amount of one or more protective compositions (e.g., UV Physical block). Note that where a tattoo removal procedure has caused injury or removal of the epidermis, the application of exfoliant may advantageously be delayed until skin has re-epithelialized (or redness has greatly subsided). In embodiments, the second treatment occurs in the evening hours and includes the topical application of an effective amount of one or more preparatory compositions (e.g., gentle cleanser, and toner) followed by application of an effective amount of one or more corrective compositions (e.g., hydroquinone and tretinoin compositions). In embodiments, tretinoin is used either alone or in combination with other corrective compositions during the evening treatment.

Kit Components

As the pre-procedure treatment regimen requires the sequential application of various components, it has also been found that kits greatly facilitate the user in performing the pre-treatment regimen consistently. One suitable kit for pre-treatment includes the following:

Foaming Gel
Toner
Obagi Nu-Derm ® Clear
Obagi Nu-Derm ® Exfoderm Forte
Blender
Healthy Skin Protection SPF 35
Tretinoin (0.1% or 0.05%)

As the treatment regimen requires the sequential application of various post procedure components, it has also been found that kits greatly facilitate the user in performing the post-treatment regimen consistently. One suitable kit for post-treatment includes the following:

Cleanser
Toner
Obagi Nu-Derm ® Clear
Obagi Nu-Derm ® Exfoderm
Blender
UV Physical Sunblock
Tretinoin (0.1% or 0.05%

Typically, kits are provided with instructions for care. For example, the instructions may direct that the corrective and supplemental compositions of the pre-procedure treatment regimen be applied as follows:

| Pre-Treatment | | First | Second |
|---|---|---|---|
| Regimen for Tattoo | | Application | Application |
| Removal Procedure | Product | (a.m.) | (p.m.) |
| Apply nickel size | Foaming Gel | X | X |
| amount of preparatory | Toner | X | X |
| composition to tattoo | | | |
| and surrounding skin, | | | |
| then wash off. | | | |
| Apply large pea size | Obagi Nu-Derm ® | X | X |
| amount of corrective | Clear | | |
| composition to tattoo | Nu-Derm ® | X | |
| and surrounding skin. | Exfoderm Forte | | |
| | Obagi Nu-Derm ® | | X |
| | Blender | | |
| | Tretinoin | | X |
| Apply pea size | Sun screen or | X | |
| amount of protective | sun block | | |
| composition to tattoo | | | |
| and surrounding skin. | | | |

The instructions may also, in embodiments, direct that the corrective and supplemental compositions of the post-treatment regimen be applied as follows:

| Post-treatment | | First | Second |
|---|---|---|---|
| Regimen for Tattoo | | Application | Application |
| Removal Procedure | Product | (a.m.) | (p.m.) |
| Apply pea size | Cleanser | X | X |
| amount of preparatory | Toner | X | X |
| composition to tattoo | | | |
| and surrounding skin, | | | |
| then wash off. | | | |
| Apply large pea size | Obagi Nu-Derm ® | X | X |
| amount of corrective | Clear | | |
| composition to tattoo | Obagi Nu-Derm ® | X | |
| and surrounding skin. | Exfoderm | | |
| | Obagi Nu-Derm ® | | X |
| | Blender | | |
| | Tretinoin | | X |
| Apply pea size | Physical UV | X | |
| amount of protective | Block | | |
| composition to tattoo | | | |
| and surrounding skin. | | | |
| Reapply as needed. | | | |

These instructions are illustrative. Those skilled in the art may readily envision other instructions. The second application may be performed at least four hours after the first treatment for both the pre-treatment and the post-treatment.

In embodiments, a patient follows a prescribed treatment regimen twice a day (in the morning and at night) for up to about nine weeks prior to undergoing tattoo removal, preferably from about one to about seven weeks prior to undergoing tattoo removal, most preferably from about three to about four weeks prior to undergoing tattoo removal. The pre-treatment regimen involves applying designated products from the commercially available Obagi Nu-Derm® system and/or prescription product in the smallest possible amount sufficient to cover at least the site intended for the tattoo removal treatment, in embodiments, the entire face of the patient even if only a small area of the face is to receive the tattoo removal treatment. The regimen may advantageously be as follows:

| | Morning | Evening |
|---|---|---|
| Prepare | Foaming Gel | Foaming Gel |
| | Toner | Toner |
| Correct | Clear 4% Hydroquinone 1 gm | Clear 4% Hydroquinone 1 gm |
| Stimulate | | Tretinoin 0.05% |
| | | Blender 4% HQ 0.5 gm |
| Protect | Sunfader | |

After the desired pre-treatment period, tattoo removal is performed. After ensuring that sufficient re-epithelialization occurs, the patient resumes treatment with the previously used treatment regimen for a post-treatment time of up to about eleven weeks, preferably about one to about nine weeks, most preferably from about four to about six weeks.

Benefits of Pre-Treatment and Optional Post-Treatment

The use of the presently described methods may provide one or more benefits to the skin of the user undergoing tattoo removal. For example, by employing the methods described herein, a patient undergoing tattoo removal may observe perioral fine wrinkle improvement, periocular fine wrinkle improvement, hyperpigmentation improvement, hypopigmentation improvement, tactile roughness improvement, sallowness improvement, acne scarring improvement and/or increased overall skin quality. Additionally, a patient undergoing tattoo removal employing the methods described herein may observe no worsening of Erythema.

### EXAMPLE 1

A 34 year old white female in good general health is presented to dermatologist requesting removal of a tattoo from her inner thigh. The patient has little sun damage and indicates the desire to minimize, reduce, or eliminate the side effects of the tattoo removal process.

Pre-treatment:

The patient is started on a pre-treatment protocol to precondition her skin in preparation for the upcoming tattoo removal procedure.

The patient is prescribed tretinoin (0.05% or 0.1%) and provided with a pre-treatment kit containing a container of foaming gel, toner, Obagi Nu-Derm® Clear formulation, Obagi Nu-Derm® Exfoderm Forte Formulation, and Obagi Healthy Skin Protection having SPF 35, blender, and prescribed tretinoin. Each container provides enough formulation in an amount sufficient to be applied to inner thigh as instructed below for between 3 to 6 weeks prior to the tattoo removal procedure.

Each kit contains instructions for the patient to apply the pre-treatment compositions every morning. The instructions require the following steps to be followed in the morning in sequential order: 1) apply a nickel-sized amount of foaming gel to wet skin, massage into the inner thigh and rinse thoroughly; 2) apply toner using cotton pads or fingertips to entire inner thigh; 3) apply 0.5 grams of Obagi Nu-Derm® Clear to inner thigh; 4) apply Obagi Nu-Derm® Exfoderm Forte to inner thigh, and to rub in thoroughly; and 5) apply Healthy Skin Protection (SPF 35) to inner thigh (Application of the protective composition can be repeated after 2 hours if patient is in direct sunlight).

The instructions further require the following steps to be followed in the evening in sequential order: 1) apply a nickel-sized amount of foaming gel to wet skin, massage into inner thigh and rinse thoroughly; 2) apply toner using cotton pads or fingertips to inner thigh; 3) apply 0.5 grams of Obagi Nu-Derm® Clear to inner thigh; 4) apply blender (0.5 grams) and tretinoin (0.5 grams). Apply in the evening after Obagi Nu-Derm Clear by measuring 0.5 grams of Blender, followed by a prescribed amount of tretinoin. Combine and apply evenly on inner thigh.

The patient performs the pre-treatment regimen in accordance with these instructions and preconditions the surface of skin in need of a tattoo removal procedure for three weeks prior to the procedure.

Treatment:

A dermatologist then performs a Q-Switched laser removal process in which the laser is pulsed quickly to dissolve the ink without destroying the live tissue in the process. This laser removal process is performed by procedures known in the art.

Post-treatment:

The patient is started on post-treatment protocol to ensure that skin heals quickly, and to reduce the likelihood of post-procedural reactions or complications like post-inflammatory hyperpigmentation, erythema (redness), acne and scarring. Post-conditioning likely extends the duration of the cosmetic benefit received.

The patient is prescribed tretinoin (0.05% or 0.1%) and provided with a post-treatment kit containing a container of gentle cleanser, toner, Obagi Nu-Derm® Clear formulation, Obagi Nu-Derm® Exfoderm formulation, and Obagi Nu-Derm® UV Physical Sunblock, blender, and prescribed tretinoin. Each container provides enough formulation in an amount sufficient to be applied to the inner thigh as instructed below for between 3 to 6 weeks after the tattoo removal procedure.

Each kit contains instructions for the patient to apply the post-treatment compositions every morning. The instructions require the following steps to be followed in the morning in sequential order: 1) apply gentle cleanser to the inner thigh, rinse with lukewarm water; 2) apply toner using fingertips to entire inner thigh (do not rinse); 3) apply 0.5 grams of Obagi Nu-Derm® Clear to inner thigh in feathering motion; 4) apply Obagi Nu-Derm® Exfoderm to entire inner thigh; 5) apply Physical UV Block to inner thigh. Application of protective composition can be repeated after 2 hours if patient is in direct sunlight.

Instructions further require the following steps to be followed in the evening in sequential order: 1) apply a nickel-sized amount of gentle cleanser to wet skin, massage into inner thigh and rinse thoroughly; 2) apply toner using cotton pads or fingertips to inner thigh; 3) apply 0.5 grams of Obagi Nu-Derm® Clear to inner thigh; 4) apply blender (0.5 grams) and tretinoin (0.5 grams). Apply in the evening after Obagi Nu-Derm® Clear by measuring 0.5 grams of Blender, followed by a prescribed amount of tretinoin. Combine and apply evenly on inner thigh.

The patient performs the post-treatment regimen in accordance with these instructions and post-conditions the surface of skin subjected to a tattoo removal procedure for three weeks after procedure.

### EXAMPLE 2

A 35 year old white male in good general health is presented to dermatologist for removal of a small tattoo from his face. The patient has little sun damage and indicates the desire to minimize, reduce, or eliminate the any side effects from the tattoo removal, especially scarring.

Pre-treatment:

The patient is started on a pre-treatment protocol to precondition his skin for upcoming tattoo removal procedure.

The patient is prescribed tretinoin (0.05% or 0.1%) and provided with a pre-treatment kit containing a container of foaming gel, toner, Obagi Nu-Derm® Clear formulation, Obagi Nu-Derm® Exfoderm Forte Formulation, and Obagi Healthy Skin Protection having SPF 35, blender, and prescribed tretinoin. Each container provides enough formulation in an amount sufficient to be applied to face as instructed below for between 3 to 6 weeks prior to the tattoo removal procedure.

Each kit contains instructions for the patient to apply the pre-treatment compositions every morning. The instructions require the following steps to be followed in the morning in sequential order: 1) apply a nickel-sized amount of foaming gel to wet skin, massage into entire face and neck and rinse thoroughly; 2) apply toner using cotton pads or fingertips to entire face; 3) apply 0.5 grams of Obagi Nu-Derm® Clear to face; 4) apply Obagi Nu-Derm® Exfoderm Forte to entire face using caution in eye area because stinging may occur, and to rub in thoroughly; and 5) apply Healthy Skin Protection (SPF 35) to face and neck (Application of protective composition can be repeated after 2 hours if patient is in direct sunlight).

The instructions further require the following steps to be followed in the evening in sequential order: 1) apply a nickel-sized amount of foaming gel to wet skin, massage into entire face and neck and rinse thoroughly; 2) apply toner using cotton pads or fingertips to entire face; 3) apply 0.5 grams of Obagi Nu-Derm® Clear to face; 4) apply blender (0.5 grams) and tretinoin (0.5 grams). Apply in the evening after Obagi Nu-Derm Clear by measuring 0.5 grams of Blender, followed by a prescribed amount of tretinoin. Combine and apply evenly on entire face, extending to the hairline. Apply around eye area as directed.

The patient performs the pre-treatment regimen in accordance with these instructions and preconditions the surface of skin having the tattoo and in need of a tattoo removal procedure for three weeks prior to the tattoo removal procedure.

Treatment:

A dermatologist then performs a Q-Switched laser removal process in which the laser is pulsed quickly to dissolve the ink without destroying the live tissue in the process. This laser tattoo removal process is performed by procedures known in the art.

Post-treatment:

The patient is started on a post-treatment protocol to ensure that skin heals quickly, and to reduce the likelihood of post-procedural reactions or complications.

The patient is prescribed tretinoin (0.05% or 0.1%) and provided with a post-treatment kit containing a container of gentle cleanser, toner, Obagi Nu-Derm® Clear formulation, Obagi Nu-Derm® Exfoderm Formulation, and Obagi Nu-Derm® UV Physical Sunblock, blender, and prescribed tretinoin. Each container provides enough formulation in an amount sufficient to be applied to face as instructed below for between 3 to 6 weeks after the tattoo removal procedure.

Each kit contains instructions for the patient to apply the post-treatment compositions every morning. The instructions require the following steps to be followed in the morning in sequential order: 1) apply gentle cleanser to face, rinse with lukewarm water; 2) apply toner using fingertips to entire face (do not rinse); 3) apply 0.5 grams of Obagi Nu-Derm® Clear to face in feathering motion; 4) apply Obagi Nu-Derm® Exfoderm to entire face; 5) apply Physical UV Block to face and neck. Application of protective composition can be repeated after 2 hours if patient is in direct sunlight.

Instructions further require the following steps to be followed in the evening in sequential order: 1) apply a nickel-sized amount of gentle cleanser to wet skin, massage into entire face and neck and rinse thoroughly; 2) apply toner using cotton pads or fingertips to entire face; 3) apply 0.5 grams of Obagi Nu-Derm® Clear to face; 4) apply blender (0.5 grams) and tretinoin (0.5 grams). Apply in the evening after Obagi Nu-Derm® Clear by measuring 0.5 grams of Blender, followed by a prescribed amount of tretinoin. Combine and apply evenly on entire face, extending to the hairline. Apply around eye area as directed.

The patient performs the post-treatment regimen in accordance with these instructions and post-conditions the surface of skin subjected to a tattoo removal procedure for three weeks after the procedure. The patient does not have adverse events such as reactions or complications.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

What is claimed is:

1. A method comprising:
preconditioning an area of skin of a subject intended to receive a tattoo removal procedure by sequential topical application of a preparatory composition and a corrective composition to the area of skin.

2. A method as in claim 1 further comprising applying a protective composition after application of the corrective composition.

3. A method as in claim 1 further comprising waiting a predetermined period of time and repeating the preconditioning.

4. A method as in claim 1 further comprising performing a tattoo removal procedure on the preconditioned area of skin.

5. A method comprising:
post-treating an area of skin of a subject that has previously been treated with a tattoo removal procedure by sequential topical application of a preparatory composition and a corrective composition to the area of skin.

6. A method as in claim 5 further comprising applying a protective composition after application of the corrective composition.

7. A method as in claim 5 further comprising waiting a predetermined period of time and repeating the post-treating.

8. A method comprising:
preconditioning an area of skin of the subject intended to receive a tattoo removal procedure by administering to the area of skin a first treatment and, after a predetermined period of time, a second treatment,
the first treatment comprising the sequential topical application of one or more preparatory compositions, a first corrective composition, and one or more protective compositions;
the second treatment comprising the sequential topical application of one or more preparatory compositions, and a second corrective composition,
wherein the second corrective composition comprises a first active ingredient sensitive to oxidation and a second active ingredient comprising a retinoid.

9. The method of claim 8 wherein the one or more preparatory compositions are selected from the group consisting of cleansers, toners and combinations thereof.

10. The method of claim 8 wherein the one or more protective compositions are selected from the group consisting of sunscreens, sun blocks, moisturizers and combinations thereof.

11. The method of claim 8 wherein the first corrective composition comprises an active ingredient that is sensitive to oxidation; a preservative; a chelating agent; an emulsifier; a humectant; a pH adjuster; an antioxidant; an emollient; a reducing agent and water.

12. The method of claim 8 wherein the first corrective composition comprises hydroquinone.

13. The method of claim 8 wherein the second corrective composition further comprises a preservative; a chelating agent; an emulsifier; a humectant; a pH adjuster; an antioxidant; an emollient; a reducing agent and water.

14. The method of claim 8 wherein the first active ingredient is hydroquinone.

15. The method of claim 8 further comprising the step of waiting at least four hours between the first treatment and the second treatment.

16. The method of claim 8 further comprising treating the skin with a tattoo removal procedure.

17. The method of claim 16 further comprising
post-treating the skin of the subject treated with a tattoo removal procedure by administering to the area of skin a first treatment and, after a predetermined period of time, a second treatment,
the first treatment comprising the sequential topical application of one or more preparatory compositions, a first corrective composition, and one or more protective compositions;
the second treatment comprising the sequential topical application of one or more preparatory compositions, and a second corrective composition,
wherein the second corrective composition comprises a first active ingredient sensitive to oxidation and a second active ingredient comprising tretinoin.

18. A method comprising
preconditioning an area of skin of the subject intended to receive a tattoo removal procedure by administering to the area of skin a first treatment and, after a predetermined period of time, a second treatment,
the first treatment comprising the sequential topical application of a cleanser, a toner, a first corrective composition containing hydroquinone, and a protective composition;
the second treatment comprising the sequential topical application of a cleanser, a toner, a second corrective composition comprising hydroquinone and a retinoid; and
performing a tattoo removal procedure on the preconditioned area of skin.

19. The method of claim 18 further comprising post-treating the area of skin subjected to the tattoo removal procedure by administering to the area of skin a first treatment and, after a predetermined period of time, a second treatment,
the first treatment comprising sequential topical application of a cleanser, a toner, a first corrective composition containing hydroquinone, and a protective composition;
the second treatment comprising sequential topical application of a cleanser, a toner, a second corrective composition comprising hydroquinone and a retinoid.

20. The method of claim 18 wherein the first pre-conditioning treatment occurs in the morning.

21. The method of claim 20 wherein second pre-conditioning treatment occurs at least four hours after the first pre-conditioning treatment.

22. The method of claim 19 wherein the first post-treating treatment occurs in the morning.

23. The method of claim 22 wherein second post-treating treatment occurs at least four hours after the first post-treating treatment.

24. A skin treatment kit for use prior to a tattoo removal procedure comprising:
a corrective composition;
at least one supplemental composition selected from the group consisting of preparatory compositions, protective compositions, and combinations thereof; and
instructions for pre-conditioning an area of skin by the ordered application of the corrective composition and the at least one supplemental compositions in preparation for a tattoo removal procedure.

25. A kit as in claim 24 wherein the corrective composition comprises: an active ingredient that is sensitive to oxidation; a preservative; a chelating agent; an emulsifier; a humectant; a pH adjuster; an antioxidant; an emollient; a reducing agent and water

26. A kit as in claim 24 wherein the at least one supplemental composition is a preparatory composition selected from the group consisting of cleansers, toners and combinations thereof.

27. A kit as in claim 24 wherein the at least one supplemental composition is a protective composition selected from the group consisting of sunscreens, sun blocks and combinations thereof.

28. A kit comprising:
a preparatory composition;
a corrective composition;
a protective composition; and
instructions for pre-conditioning an area of skin in preparation for a tattoo removal procedure by administering to the area of skin a first treatment and, after a predetermined period of time, a second treatment,
the first treatment comprising the sequential topical application of the preparatory composition, the corrective composition, and the protective composition;
the second treatment comprising the sequential topical application of the preparatory composition, and the corrective composition.

29. A skin treatment kit for use after a tattoo removal procedure comprising:
a corrective composition;
at least one supplemental composition selected from the group consisting of preparatory compositions, protective compositions, and combinations thereof; and
instructions for post-treating an area of skin by the ordered application of the corrective composition and the at least one supplemental compositions after a tattoo removal procedure.
